(19) United States
(12) Patent Application Publication
(10) Pub. No.: US 2009/0169921 A1
(43) Pub. Date: Jul. 2, 2009

(54) SYNTHESIS OF TRIPHENYLENE AND PYRENE BASED AROMATICS AND THEIR APPLICATION IN OLEDS

(76) Inventors: Chien-Hong Cheng, Hsin-Chu City (TW); Chang-Sheng Lin, Taoyuan County (TW)

Correspondence Address: THOMAS, KAYDEN, HORSTEMEYER & RISLEY, LLP, 600 GALLERIA PARKWAY, S.E., STE 1500, ATLANTA, GA, 30339-5994, US

(21) Appl. No.: 11/968,353

(22) Filed: Jan. 2, 2008

(51) Int. Cl. C09K 11/00; B32B 9/00; C07C 13/66; C07D 333/10

(52) U.S. Cl. 428/690; 585/26; 549/29

(57) ABSTRACT

The present invention provides a compound of the general formula

Ar¹—R¹—Ar² (I)

wherein Ar¹ and Ar² independently represent triphenylenyl or pyrenyl, and R¹ represent a bond, aryl, or heteroaryl. The present invention also provides a process for the preparation of the compound formula (□), and an organic electroluminescence device utilizing luminescent material comprising the compound of formula (□) as an emitting layer.

### BACKGROUND OF THE INVENTION

1. Field of the Invention

The invention relates to a novel compound, which exhibits good thermal stability and high emitting efficiency. More particularly, the invention relates to a compound for serving as an emitting layer for organic electroluminescence devices, especially in the blue to green spectrum.

2. Description of the Related Art

The earliest report of organic electroluminescence was made by Pope et al in 1963, who observed a blue fluorescence from 10-20 □m of crystalline anthracene by applying voltage across opposite sides of the crystal. Thus, starting a wave of first improvements in organic electroluminescence research. However, difficulties of growing large areas of crystals were a challenge. The driving voltage of the device was too high and the efficiency of organic materials was lower than inorganic material. Because of the disadvantages of the devices, the devices were not widely applied due to practical purposes.

The next major development in organic electroluminescence devices was reported in 1987. Tang and VanSlyke of Eastman Kodak Company used vacuum vapor deposition and novel heterojection techniques to prepare a multilayered device with hole/electron transporting layers. 4,4-(cyclohexane-1,1-diyl)bis(N,N-dip-toylbenzenamine) (TPAC) was used as a hole transporting layer, and Alq3 (tris(8-hydroxyquinolinato) aluminum(□)) film with good film-forming properties was used as an electron transporting and emitting layer. A 60-70 nm-thick film was deposited by vacuum vapor deposition with a low-work function Mg:Ag alloy as the cathode for efficient electrons and holes injection. The bi-organic-layer structure allowed the holes and electrons to recombine at the p-n interface and then emit light. The device emitted green light of 520 nm, and is characterized by low driving voltage (<10 V), high quantum efficiency (>1%) and good stability. The improvements arouse great interest in the organic electroluminescence technique.

Meanwhile, Calvendisg and Burroughes et al. at Cambridge University in 1990 reported the first research using conjugated polymer TAPC(4,4′-(cyclohexane-1,1-diyl)bis(N,N-dip-tolylbenzenamine)) as an emitting layer in a single-layered device structure by solution spin coating. The development of an emitting layer with conjugated polymer drew great interest and quickly sparked research due to the simplicity of fabrication, good mechanical properties of polymer, and semiconductor-like properties. In addition, a large number of organic polymers are known to have high fluorescence efficiencies.

The basic mechanism of organic electroluminescence involves the injection of the carrier, transport, recombination of carriers and exciton formed to emit light. The general structure of organic electroluminescence device includes an anode, a hole transporting layer (HTL), an emitting layer (EML), an electron transporting layer (ETL) and a cathode. For choosing materials, high work function and transport indium tin oxide (ITO) was chosen as anode, N,N′-diphenyl-N, N′-bis-(3-methylphenyl)-1,1′biphenyl-4,4′-diamine (TPD) or N, N′-bis-phenyl-N, N′-bis-(1-naphthyl)-1,1′-biphenyl-4,4′-diamine (NPB) was used as hole transporting layer, Alq and 2-2′-2″-(1,3,5-benzenetryl)tris-(1-phenyl-1-H-benzimi-dazole) (TPBI) were used as electron transporting layer, and Ca with low work function, Mg: Ag alloy, LiF/Al alloy and Li/Al were used as cathode. Then, all materials were deposited by thermal evaporation in series of hole transporting layer, emitting layer, electron transporting layer, and finally the cathode. If the energy gap between the ITO electrode and the hole transporting layer was too large, two problems occurred: 1) hole injection was difficult, and 2) hole transporting had low efficiency. In order to solve the problems, a layer of hole injection material was added to reduce the energy gap between the ITO electrode and hole transport layer. Consequently, the holes were readily injected from the ITO electrode to the hole transporting layer. CuPc and poly (3,4-ethylenedioxythiophene):poly (styrene sulfonate) are often used as hole injection material.

When two electrodes of a device are positively biased, electrons will be injected from a cathode into a LUMO (low lowest unoccupied molecular orbital) and holes will be injected from an anode into a HOMO (highest occupied molecular orbital). By the driving force of the external electric field, holes move to the cathode and electrons move to the anode. When the electrons recombine with holes in the emitting layer, excitons are formed and then emit light.

If a hole blocking layer is added between the emitting layer and the electron transporting layer, it can prevent the excess holes from moving to the cathode to neutralize the electrons.

In research of blue-emitting materials based on small molecular, Dr. Shih of the National Tsing Hua University successfully synthesized 2,2′-bistriphenylene (BTP) as a blue-emitting material with high melting point and good efficiency. The BTP was synthesized by dimerization of epoxide and catalyzed by palladium complex. For device ITO/TPD /BTP/TPBI/Mg: Ag, showed an emitting light at 458 nm, the external quantum efficiencies was up to 4.2%, the maximum current, power, and brightness efficiencies were up to 4.2%, 4.0 cd/A , and 2.5 Im/W, respectively. A turn-on voltage was 3.5 V, and the full-width at half maximum was only 72 nm. The CIE coordinates were maintained to be (015, 0.28), almost independent of the external applied voltage.

In addition to BTP, Wu and Dr. Ku of the National Tsing Hua University demonstrated a series of pyrene-based blue-emitting material. They synthesized nine derivatives. Among the various derivatives, 1,1′-(2,5-dimethoxy-1,4-phenylene)dipyrene (P2) with glass-transition temperature of 133°C. had the best performance. For a device composed of ITO/TPD /P2/TPBI/Mg: Ag, showed an emitting light at 488 nm, a turn-on voltage of 3.0 V, the external quantum efficiencies over theoretic limiting values up to 6.1%, the maximum brightness, current, and power efficiencies were up to 74590 cd/m², 12.6 cd/A and 6.7 Im/W, respectively. The CIE coordinates were calculated to be (015, 0.28). The emitting color was sky-blue.

Professor Wong and Wu of National Chiao Tung University in 2004 used the derivatives of ter(9,9-diarylfluorene)s (TDAFs) as the blue-emitting material. Because of the strong binding energy of the Cₛₚ₃-Cₛₚ₂ structure, the film of the spiro structure had high thermal endurance. For a device composed of ITO/PEDOT:PSS/TDAF1/TPBI/LiF/Al, a turn-on voltage of 2.5 V resulted, the current and brightness were 1.53 cd/A and 14000 cd/m², respectively. The CIE coordinates were calculated to be (016, 0.24). Although the device exhibited high external quantum efficiencies of 5.3%, unfortunately the TDAF1 was the only one with non-Tg (glass transition temperature) among the three TDAFs.

Professor Shu of the National Chiao Tung University and Professor Tao of the Academia Sinica in 2005 co-reported a compound of 2,7-bis(2,2-diphenylvinyl)9,9′-spirobifluorene (DPVSBF) derived from 4,4′-bis(2,2-diphenylvinyl)-1,1′-biphenyl (DPVBi). The main change was that the original biphenyl structure was changed to a spirobifluorene structure. As a result, the glass transition temperature was raised from 64° C. to 115° C., which substantially improved the thermal stability of the film. For a device composed of ITO/NPB/DPVSBF/Alq/LiF/Al, an emitting light at 474 nm resulted, the external quantum, brightness, current, and power efficiencies were 3.03%, 41247 cd/m², 5.33 cd/A, and 4.76 Im/W, respectively. The CIE coordinates were calculated to be (016, 0.24). Not only were the efficiencies and brightness of DPVSBF-based device better than DPVBi-based device, but also the lifetime of DPVSBF-based device improved 16 times of that of DPVBi-based device.

Professor Li of the City University of Hong Kong also reported a blue-emitting material combing pyrene and fluorine. The 2,7-dipyrenyl-9,9′-dimethyl-fluorene (DPF) derivatives all exhibited high glass transition temperature (T_{g}), between 145° C. and 193° C. The device based on DPF had the best performance. A device composed of ITO/CuPc/NPB/DPF/TPBI/LiF/Mg:Ag, showed an emitting light at 469 nm, the current, power, and maximum brightness efficiencies were 5.3 cd/A, 3.0 Im/W, and 9260 cd/m², respectively. The CIE coordinates were calculated to be (016, 0.22).

According to the above reference, the efficiency of device is independent of the number of benzyl group (conjugated group). Increasing steric hinderance indeed raises the glass transition temperature.

In previous work of the inventor, triphenylene derivatives were prepared as blue-emitting layer and it was found that the material exhibited good performance. However, these derivatives had no glass transition temperature, and they suffered from thermal instability. Recently, research on pyrenyl derivatives has been reported. It was found that a portion of pyrenyl derivatives had good glass transition temperature and the derivatives itself exhibited good quinine sulfate equivalent (Q. E.) (71%). It is possible to improve the efficiency of devices by varying the number of central benzyl group of pyrenyl derivative. In addition, the emitting wavelength can be altered by varying conjugated lengths of compounds.

Sato et al. reported an improved hole transporting material with more □-electron groups and heavy atoms for reducing rotational moment to raise the glass transition temperature. Professor Shirota reported another material by adding rigid fluorine to raise the glass transition, but excess of thiophene made the emitting light produce red-shift.

Wong's research group of the National Taiwan University in 2002 reported fluorine derivatives based on oligothiophene as core chromophores. By varying conjugation lengths of central thiophene, the emitting color of the molecular changed from light blue to bright yellow. The result also conforms to the report of professor Shirota. Another important issue was that the material exhibited stable glass transition temperature of 153° C.˜154°C., irrespective of the conjugation lengths of the oligothiophene.

It is important to seek excellent electroluminescence materials in the wavelength of blue to green region in order to make the devices exhibit high performance, good thermal stability and high emitting efficiency. According to the reasons described above, pyrenyl and thrphrntlenyl asymmetric derivative were selected to be used as an emitting layer in the present inventions.

### BRIEF SUMMARY OF THE INVENTION

An objective of the present invention is to provide a novel compound as an emitting layer for organic electroluminescence devices. The organic electroluminescence device (OLED) shows high brightness, high external quantum and current efficiency, and excellent power efficiency due to the good thermal stability and high emitting efficiency of the compound.

A further objective of the present invention is to provide a process of preparing the above-mentioned compound.

An yet a further objective of the present invention is to provide OLED devices, which comprise an anode, a hole transporting layer, an emitting layer, an electron transporting layer, and a cathode, wherein the OLED devices utilize luminescent material comprising the compound of the invention as an emitting layer.

The present invention provides a novel compound of formula (□):

Ar¹—R¹—Ar2 (I),

wherein Ar¹ and Ar² independently represent triphenylenyl or pyrenyl and R¹ represent a bond, aryl or heteroaryl.

### DETAILED DESCRIPTION OF THE INVENTION

The present invention provides a novel compound of formula (□):

Ar¹—R¹—Ar2 (I),

wherein Ar¹ and Ar² independently represent triphenylenyl or pyrenyl and R¹ represent a bond, aryl or heteroaryl.

Ar¹, Ar² and R¹ independently comprise one or more substituents; preferably they comprise one, two, three, or four substituents. The substituents are selected from the group consisting of: hydrogen, halogen (fluorine, chlorine, bromine, iodine); aryl, halogen-substituted aryl, halogen-substituted aryl alkyl, haloalkyl-substituted aryl, haloalkyl-substituted aryl alkyl, aryl-substituted C1-C20 alkyl; electron donating group, such as C1-C20 alkyl (methyl, ethyl, butyl), C1-C20 cycloalkyl (cyclohexyl), C1-C20 alkoxy, C1-C20-substituted amino group, substituted aryl amino group (aniline); electron withdrawing group, such as halogen, nitrile, nitro, carbonyl, cyano (—CN), halogen-substituted C1-C20 alkyl(trifluoromethyl-); and heterocyclo-substituted group.

The aryl group includes but is not limited to phenyl, naphthyl, diphenyl, anthryl, pyrenyl, phenanthryl, fluorine or other fused polycyclic phenyl.

The heteroaryl group includes but is not limited to pyrane, pyrroline, furan, benzofuran, thiophene, benzothiophene, pyridine, quinoline, isoquinoline, pyrazine, pyrimidine, pyrrole, pyrazole, imidazole, indole, thiazole, isothiazole, oxazole, isoazole, benzothiazole, benzoxazole, 1,2,4-triaole, 1,2,3-triazole, phenanthroline or other heteroaryl.

In one embodiment of the above-mentioned formula (□), R¹ is heteroaryl, and Ar¹ and Ar² are the same.

In one embodiment, the compound has the formula shown below, wherein R¹ is a bond:

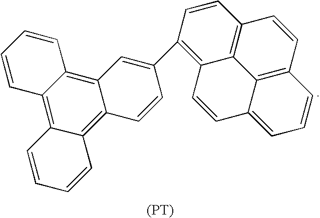

In another embodiment, the compound has the formula shown below, wherein R¹ is phenyl, and Ar¹ is different from Ar²:

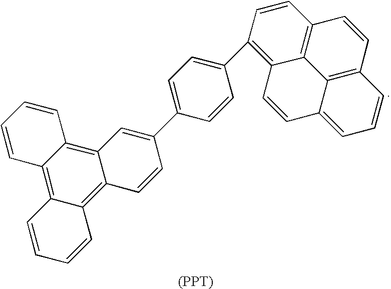

In another embodiment, the compound has the formula shown below, wherein R¹ is biphenyl, Ar¹ is different from Ar²:

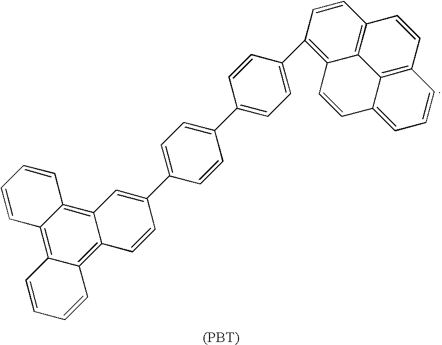

In another embodiment, the compound has the formula shown below, wherein R¹ is thiophene, Ar¹ and Ar² are triphenylene:

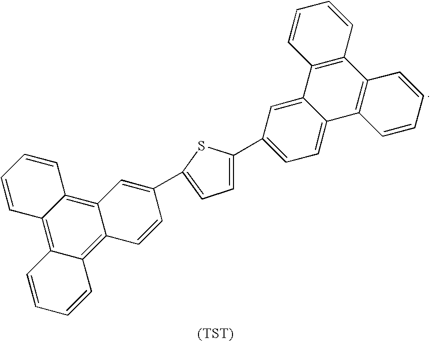

In another embodiment, the compound has the formula shown below, wherein R¹ is thiophene, and Ar¹ and Ar² are pyrenyl:

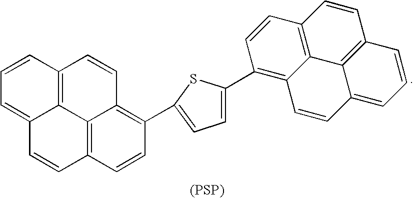

In another embodiment, the compound has the formula shown below, wherein R¹ is thiophene, and Ar¹ is different from Ar²:

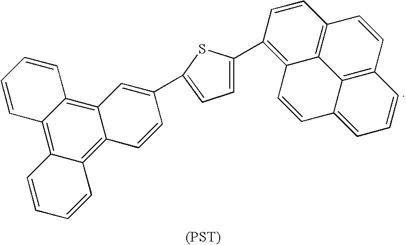

The present invention further provides a process of preparing the above-mentioned formula (□), comprising:

(a) reacting a compound of formula (□) with a compound of formula (□) to result in the compound of formula (□) when R¹ is a bond,

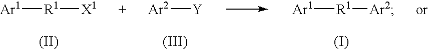

(b) reacting a compound of formula (□) with a compound of formula (□)to result in the compound of formula (□) when R¹ is aryl or heteroaryl and Ar¹ is different from Ar²;
(c) reacting a compound of formula (□) with a compound of formula (□)to result in the compound of formula (□) when R¹ is aryl or heteroaryl, Ar¹and Ar² are triphenylenyl,

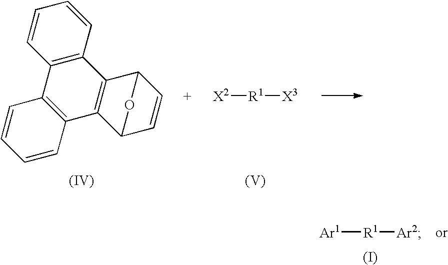

(d) reacting a compound of formula (□) with a compound of formula (□)to result in the compound of formula (□) when R¹ is aryl or heteroaryl, Ar¹and Ar² are pyrenyl,

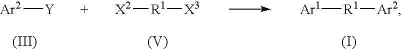

wherein X¹, X² and X³ are chlorine (Cl), bromine (Br) or iodine (I), Y is boron hydroxide (B(OH)₂).

For the above-mentioned process, the step (a), (b) and (d) are carried out by Suzuki coupling reaction, and the step (c) is carried out by a coupling reaction. The reaction conditions of Suzuki Coupling reaction or coupling reaction are well known in the art and are suitable for the processes of the present invention. The compound (□) in step (b) is produced by reacting with a compound of formula (□),

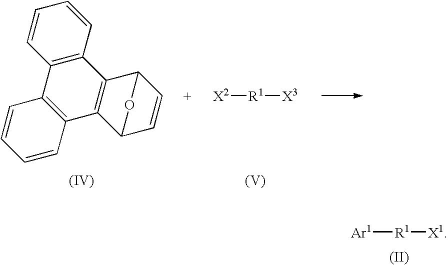

The present invention also provides organic electroluminescence devices, which comprise an anode, a hole transporting layer, an emitting layer, an electron transporting layer, and a cathode, wherein the organic electroluminescence device utilizes luminescent material comprising the compound of formula (□) as an emitting layer.

While the invention has been described by way of example and in terms of the preferred embodiments, it is to be understood that the invention is not limited to the disclosed embodiments. To the contrary, it is intended to cover various modifications and similar arrangements (as would be apparent to those skilled in the art). Therefore, the scope of the appended claims should be accorded the broadest interpretation so as to encompass all such modifications and similar arrangements.

### EXAMPLE 1

### SYNTHESIS OF COMPOUND (□) (1,4-DIHYDRO-1,4-EPOXYTRIPHENYLENE)

25.7 g (100 mmol) of 9-bromophenathalene and 11.7 g (300 mmol) of sodium amide were placed in a 500 ml reaction bottle. Vacuum was developed in the reaction bottle then nitrogen was introduced into the reaction bottle, and this cycle was repeated a few times. 49.6 g (508 mmol) of furan and 200 ml of anhydrous tetrahydroxyfuran (THF) was introduced into the reaction bottle. The mixture slowly heated to 65° C. for 6 hours. Upon completion of the reaction, the reaction mixture was filtered in order to remove the salt. The filtrate was concentrated on a rotary evaporator, and the resulting solid product was purified by separation with a silica gel column. The eluent used a mixed solvent of ethylacetate: hexane=1:5. After separation, a pale yellow solid product in 80% yield was obtained.

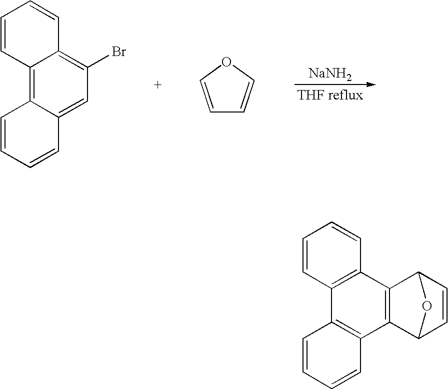

### EXAMPLE 2

### SYNTHESIS OF PYREN-1-YL-1-BORONIC ACID

2.0 g (7.12 mmol) of 1-bromopyrene was dissolved in the anhydrous THF (100 ml) and anhydrous ether (100 ml). n-Butyllithium (4.9 ml, 7.83 mmol) was slowly dripped into the solution at −78° C. in nitrogen. The color of the solution changed from a slightly transparent yellow to light and opaque yellow solution. The solution was kept at −78° C. for ten minutes, −10° C. for ten minutes, and then −78° C. for thirty minutes. Tri-methyl borate (4.93 ml, 21.36 mmol) was slowly dipped into the solution and stirred at −78° C. for thirty minutes. The color of the solution became transparent yellow-orange. Then after keeping the solution at 0° C. for three hours, the color became transparent yellow. Finally, the solution underwent reaction at room temperature for 1.5 days. Next, 100 ml of hydrochloride aqueous solution (10%) was added into the reaction bottle and the mixture was stirred vigorously for one hour. The organic layer was extracted by ethyl ester, the water layer was then extracted by ethyl ester (2×25 ml). The combined organic solution was dried over MgSO₄, and then concentrated on a rotary evaporator to obtain 1.43 g of a pale yellow solid in 80% yield.

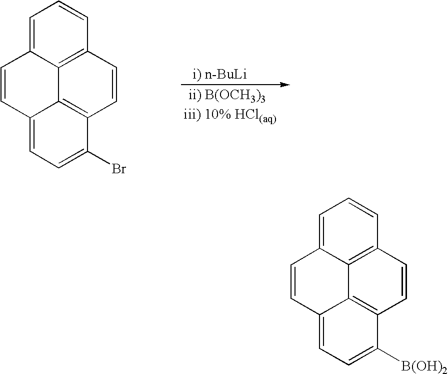

### EXAMPLE 3

### SYNTHESIS OF ASYMMETRIC COMPOUND

1.1 eq. of 1,4-dihydro-1,4-epoxytriphenylene and 1 eq. of para-(bromo-iodo)aryl compound were dissolved in toluene under the catalysts of PdCl₂(PPh₃) and reduction agent of 5 eq. of triethylamine (TEA) and 5 eq. of zinc powder. The mixed solution was kept at 110° C. and stirred for one day. Thereafter, the reaction mixture was filtered in order to remove the salt. The filtrate was concentrated on a rotary evaporator, and the resulting solid product was purified over a silica gel column. Using a mixed solvent of ethylacetate: hexane (1:5) as an eluent, a white solid bromide in 78%˜91% yield was provided.

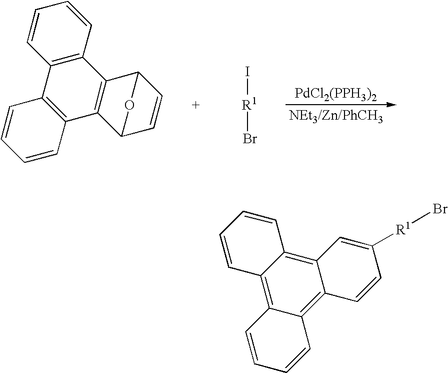

2. 1.1 eq. of 1-pyrenyl boronic acid and 1 eq. of bromo(triphenylen-2-yl) aryl were dissolved in toluene under the catalysts of Pd(PPh₃)₄ (5 mol %) and alkali agent of potassium carbonate (2 M). The volume ratio of toluene and potassium carbonate was 3:1. Suzuki Coupling reaction with C—C bond adding reaction was performed on the mixed solution. The solution was kept at 110° C. for 1 to 3 days. The yield was 71%˜88%.

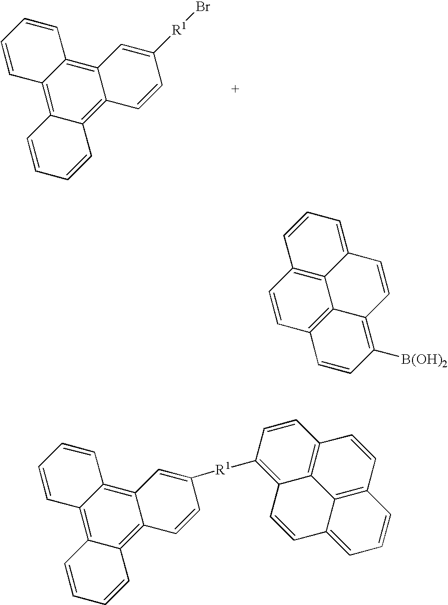

3. The crude product was purified twice by sublimation. The pressure of the sublimation was lower than 1×10-6 Torr, and the temperature of sublimation was dependent on the product. For synthesis of PT, PPT and PBT, the temperature of sublimation was 250° C.˜350° C. and for synthesis of TST, PSP and PST, the temperature of sublimation was 250° C˜310. Various physical determinations, including UV-Vis adsorption spectrum, photoluminescent (PL) emission spectrum, Differential Scanning Calorimetry (DSC), HOMO/LUMO (AC-□) and quantum efficiency was performed on the product obtained from the sublimation process. The data of these compounds were shown as in Table 1 and Table 2.

TABLE 1
the photo-physical properties of PT, PPT, PBT, TST, PSP,
PST-(□)

| | □ₘₐₓ^{a} | | | | | |
|---|---|---|---|---|---|---|
| | Abs in | □ₘₐₓ^{b} EM | □ₘₐₓEM | | | |
| | toluene | in toluene | (thin film) | HOMO^{c} | LUMO | Eg |
| compounds | (nm) | (nm) | (nm) | (ev) | (ev) | (ev) |
| PT | 346 | 404 | 460, 480 | 5.81 | 2.71 | 3.10 |
| PPT | 350 | 424 | 460 | 5.73 | 2.78 | 2.95 |
| PBT | 346 | 417 | 458 | 5.68 | 2.73 | 2.95 |
| TST | 370 | 420, 444 | 498 | 5.49 | 2.60 | 2.89 |
| PSP | 380 | 477 | 526 | 5.29 | 2.70 | 2.59 |
| PST | 372 | 482 | 514 | 5.34 | 2.70 | 2.64 |

^{a}For UV-Vis adsorption spectrum, the concentration of the solution is 1 × 10⁻⁵ M.
^{b}For photoluminescent (PL) emission spectrum, the concentration of the solution is 1 × 10⁻⁵ M.
^{c}HOMO was detected by AC-□.

TABLE 2
the photo-physical properties of PT, PPT, PBT, TST, PSP,
PST-(□)

| | | | | Quantum yield^{c} |
|---|---|---|---|---|
| compounds | Tg (° C.)^{a} | Tc (° C.)^{a} | Tm (° C.)^{a} | (%) |
| PT | 110 | NA^{b} | 255 | 95 |
| PPT | 115 | NA^{b} | 223 | 97 |
| PBT | 135 | 170 | 273 | 99 |
| TST | NA^{b} | NA^{b} | 338 | 47 |
| PSP | 80 | 131 | 232 | 30 |
| PST | 105 | 144 | 214 | 42 |

^{a}the heating rate and cooling rate individually were 10° C./min and 20° C./min.
^{b}NA = no data was detected
^{c}7-diethylamino-4-methyl-coumarin was used
d. T_{c}: the temperature of crystalline structure
e. Tₘ: the temperature of melting point

The NMR Data

PT [2-(pyren-1-yl)triphenylene]

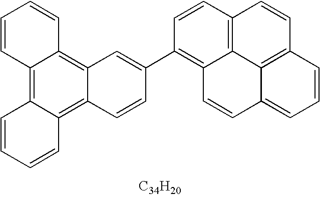

d[ppm] 9.02 (s, 1H), 8.95(d, 1H, J=8.5 Hz), 8.87-8.85(m, 1H), 8.81-8.77(m, 4H), 8.35 (d, 1H, J=8.5 Hz), 8.30(d, 1H, J=9.5 Hz), 8.25(d, 1H, J=8 Hz), 8.22-8.15(m, 4H), 8.09(d, 1H, J=9.5 Hz), 8.03 (t, 1H, J=8 Hz), 7.96(d, 1H, J=8 Hz)

13 C NMR(125 MHZ, d-THF) d[ppm] 141.06, 140.67, 138.66, 132.57, 132.08, 131.29, 130.92, 130.67, 130.47, 130.10, 130.04, 129.90, 129.65, 129.54, 128.69, 128.31, 128.23, 128.19, 128.13, 127.68, 127.37, 126.90, 126.24, 126.03, 125.96, 125.81, 125.73, 125.56, 124.93, 124.60, 124.42, 124.36, 124.30, 124.27, 122.81.
HRMS(EI+): calcd 428.1565, formed 428.1564.
Elem Anal: Calce C 95.30%, H4.70%, found C94.38%, H4.60%.

PPT [1-(pyren-1-yl)-4-(triphenylen-2-yl)benzene]

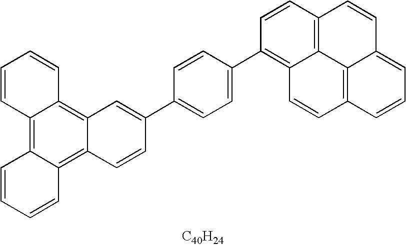

1H NMR (500 Mhz,d-THF) d[ppm] 9.16 (s, 1H), 8.98-8.95(m, 1H), 8.91-8.87(m, 1H), 8.81-8.74(m, 3), 8.32-8.20 (m, 4H), 8.16-8.01(m, 7H), 7.89(d, 1H, J=8 Hz), 7.82(d, 1H, J=8 Hz), 7.71-7.65(m, 5H)

13 C NMR(125 MHZ, d-THF) ppm. 141.50, 141.43, 140.88, 138.37, 138.32, 137.77, 137.43, 132.56, 132.06, 131.93, 131.77, 131.26, 131.11, 130.87, 130.75, 130.65, 130.08, 129.88, 129.66, 129.44, 129.33, 129.21, 128.36, 128.22, 128.15, 127.52, 126.96, 126.89, 126.03, 125.91, 125.82, 125.72, 125.60, 125.02, 124.69, 124.42, 124.29, 213.09, 122.46.
HRMS(EI+): calcd 504.1878, found 504.1881.

PBT [4-(pyren-1-yl)-4′-(triphenylen-2-yl) biphenyl]

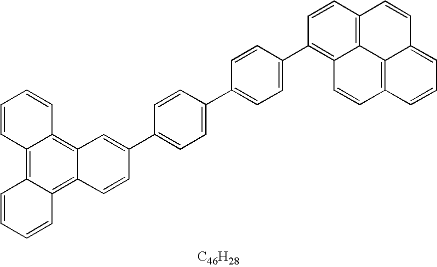

1H NMR (500 Mhz,d-THF) ppm. 9.11 (s, 1H), 8.96-8.93(m, 1H), 8.87(d, 1H, J=8 Hz), 8.79-8.68(m, 2H), 8.31-8.21 (m, 4H), 8.15-7.93(m,10H), 7.88-7.58(m, 9H).

HRMS(EI+): calcd 580.2191, found 580.2200.
Elem Anal: Calce C 95.14%, H 4.86%, found C 94.80%, H 5.19%.

PST [2-(pyren-1-yl)-5-(triphenylen-2-yl) thiophene]

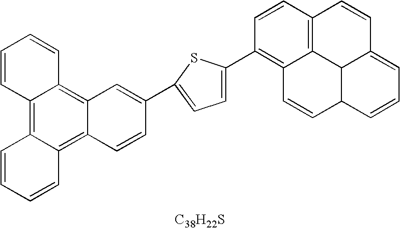

1H NMR (500 Mhz,d-THF) ppm. 9.11(s, 1H), 8.90-8.88(m, 1H), 8.32(d, 1H, J=9 Hz), 8.78-8.74(m, 3H), 8.68(d, 1H, J=9 Hz), 8.30-8.14(m, 6H), 8.10-8.03(m, 2H), 7.88(d, 1H, J=3 HZ), 7.74-7.64(m, 4H), 7.51(d,1H, J=3 Hz), 7.39(s,1H).

¹³C NMR (125 MHz, d-THF) ppm. 146.08, 143.15, 134.12, 132.56, 132.20, 132.03, 131.31, 131.18, 130.81, 130.77, 130.56, 130.53, 130.22, 129.80, 129.77, 129.63, 129.22, 128.90, 128.70, 128.39, 128.17, 128.64, 127.09, 126.32, 126.03, 125.99, 125.61, 125.33, 125.18, 124.71, 124.35, 124.28, 123.35, 123.10, 122.91, 120.70.

HRMS(EI+): calcd 510.1442, found 510.1445.
Elem Anal: calcd C 89.03%, H 4.72%, S 6.25%, found C 89.25%, H 4.56%, S 6.11%.

### EXAMPLE 4˜64

Example 4˜64 are examples using the novel present invention as an emitting layer for organic electroluminescence devices. The present invention relates to an organic electroluminescence device, which comprises an anode, a hole transporting layer, an emitting layer, an electron transporting layer, and a cathode. Between the anode and the hole transporting layer, a hole injection layer may be inserted, and between the light emitting layer and the hole transporting layer, a hole blocking layer may be inserted. ITO was used as anode, and CuPc, PEDOT:PSS, 4,4′,4″-tris(3-methylphenyl(phenyl)amino) triphenylamine (m-NTDATA) were used as a hole injection layer. NPB and TPD were used as a hole transporting layer and 2,9-dimethyl-4,7-diphenyl-1,10-phenanthroline (BCP), aluminum(□)bis(2-methyl-8-quinolinato)4-phenylphenolate (BAlq) and TPBI were used as a hole blocking layer. Alq and TPBI were used as a electron transporting layer and Mg:Ag alloy or LiF/Al was used as a cathode.

Example 4: pt-1: ITO/NPB(50 nm)/PT(30 nm)/TPBI(40 nm)/Mg:Ag(55 nm)/Ag(100 nm)

Example 5: pt-2: ITO/NPB(50 nm)/PT(30 nm)/TPBI(40 nm)/LiF(1 nm)/Al(100 nm)

Example 6: pt-3: ITO/NPB(50 nm)/PT(30 nm)/TPBI(10 nm)/Alq(30 nm)/Mg: Ag(55 nm)/Ag(100 nm)

Example 7: pt-4: ITO/NPB(50 nm)/PT(30 nm)/TPBI(10 nm)/Alq (30 nm)/LiF(1 nm) /Ag(100 nm)

Example 8: pt-5: ITO/NPB(50 nm)/PT(30 nm)/PCB(10 nm)/Alq(30 nm)/Mg:Ag(55 nm)/Ag(100 nm)

Example 9: pt-6: ITO/NPB(50 nm)/PT(30 nm)/BAlq(10 nm)/Alq(30 nm)/Mg:Ag(55 nm)/Ag(100 nm)

Example 10: pt-7: ITO/CuPc(10 nm)/NPB(50 nm)/PT(30 nm)/TPBI(40 nm)/LiF(1 nm)/Al(100 nm)

Example 11: pt-8: ITO/TPD(50nm)/PT(30nm)/TPBI(40 nm)/Mg:Ag(55 nm)/Ag(100 nm)

Example 12: pt-9: ITO/TPD(50nm)/PT(30nm)/TPBI(40 nm)/LiF (1 nm)/Ag(100 nm)

Example 13: ppt-1: ITO/NPB(50 nm)/PPT(30 nm)/TPBI(40 nm)/Mg:Ag(55 nm)/Ag(100 nm)

Example 14: ppt-2: ITO/NPB(50 nm)/PPT(30 nm)/TPBI(40 nm)/LiF(1 nm)/Ag(100 nm)

Example 15: ppt-3: ITO/NPB(50 nm)/PPT(30 nm)/TPBI(10 nm)/Alq(30 nm)/Mg:Ag(55 nm)/Ag(100 nm)

Example 16: ppt-4: ITO/NPB(50 nm)/PPT(30 nm)/BCP(10 nm)/Alq(30 nm)/Mg:Ag(55 nm)/Ag(100 nm)

Example 17: ppt-5: ITO/CuPc(10 nm)/NPB(50 nm)/PPT(30 nm)/TPBI(40 nm)/LiF(1 nm)/Al(100 nm)

Example 18: ppt-6: ITO/TPD(50 nm)/PPT(30 nm)/TPBI(40 nm)/Mg:Ag(55 nm)/Ag(100 nm)

Example 19: ppt-7: ITO/TPD(50 nm)/PPT(30 nm)/TPBI(40 nm)/LiF(1 nm)/Al(100 nm)

Example 20: ppt-8: ITO/TPD(50 nm)/PPT(30 nm)/TPBI(10 nm)/Alq(30 nm)/Mg:Ag(55 nm)/Ag(100 nm)

Example 21: ppt-9: ITO/TPD(50 nm)/PPT(30 nm)/TPBI(10 nm)/Alq(30 nm)/LiF(1 nm)/Al(100 nm)

Example 22: ppt-10: ITO/CuPc(10 nm)/TPD(50 nm)/PPT(30 nm)/TPBI(40 nm)/LiF(1 nm)/Al(100 nm)

Example 23: pbt-1: ITO/NPB(50 nm)/PBT(30 nm)/TPBI(40 nm)/Mg:Ag(55 nm)/Ag(100 nm)

Example 24: pbt-2: ITO/NPB(50 nm)/PBT(30 nm)/TPBI(40 nm)/LiF(1 nm)/Al(100 nm)

Example 25: pbt-3: ITO/NPB(50 nm)/PBT(30 nm)/TPBI(10 nm)/Alq(30 nm)/Mg:Ag(55 nm)/Ag(100 nm)

Example 26: pbt-4: ITO/NPB(50 nm)/PBT(30 nm)/TPBI(10 nm)/Alq (30 nm)/LiF(1 nm)/Al(100 nm)

Example 27: pbt-5: ITO/NPB(50 nm)/PBT(30 nm)/BCP(10 nm)/Alq(30 nm)/Mg:Ag(55 nm)/Ag(100 nm)

Example 28: pbt-6: ITO/CuPc(10 nm)/NPB(50 nm)/PBT(30 nm)/TPBI(40 nm)/Mg:Ag(55 nm)/Ag(100 nm)

Example 29: pbt-7: ITO/CuPc(10 nm)/NPB(50 nm)/PBT(30 nm)/TPBI(40 nm)/LiF(1 nm)/Al(100 nm)

Example 30: pbt-8: ITO/TPD(50 nm)/PBT(30 nm)/TPBI(40 nm)/Mg:Ag(55 nm)/Ag(100 nm)

Example 31: pbt-9: ITO/ TPD(50 nm)/PBT(30 nm)/TPBI(40 nm)/LiF(1 nm)/Al(100 nm)

Example 32: pbt-10: ITO/ TPD(50 nm)/PBT(30 nm)/TPBI(10 nm)/Alq(30 nm)/Mg:Ag(55 nm)/Ag(100 nm)

Example 33: pbt-11: ITO/ TPD(50 nm)/PBT(30 nm)/TPBI(10 nm)/Alq(30 nm)/LiF(1 nm )/Al(100 nm)

Example 34: pbt-12: ITO/CuPc(10 nm)/TPD(50 nm)/PBT(30 nm)/TPBI(40 nm)/Mg:Ag(55 nm)/Ag(100 nm)

Example 35: pbt-13: ITO/CuPc(10 nm)/TPD(50 nm)/PBT(30 nm)/TPBI(40 nm)/LiF(1 nm)/Al(100 nm)

Example 36: tst-1: ITO/NPB(50 nm)/TST(30 nm)/TPBI(40 nm)/Mg:Ag(55 nm)/Ag(100 nm)

Example 37: tst-2: ITO/NPB(50 nm)/TST(30 nm)/TPBI(40 nm)/LiF(1 nm)/Al(100 nm)

Example 38: tst-3: ITO/NPB(50 nm)/TST(30 nm)/TPBI(10 nm)/Alq(30 nm)/Mg:Ag(55 nm)/Ag(100 nm)

Example 39: tst-4: ITO/NPB(50 nm)/TST(30 nm)/BCP(10 nm)/Alq(30 nm)/Mg:Ag(55 nm)/Ag(100 nm)

Example 40: tst-5: ITO/CuPc(10 nm)/NPB(50 nm)/TST(30 nm)/TPBI(40 nm)/Mg:Ag(55 nm)/Ag(100 nm)

Example 41: tst-6: ITO/CuPc(10 nm)/NPB(50 nm)/TST(30 nm)/TPBI(40 nm)/ LiF(1 nm)/Al(100 nm)

Example 42: tst-7: ITO/TPD(50 nm)/TST(30 nm)/TPBI(40 nm)/Mg:Ag(55 nm)/Ag(100 nm)

Example 43: tst-8: ITO/TPD(50 nm)/TST(30 nm)/TPBI(40 nm)/LiF(1 nm)/Al(100 nm)

Example 44: tst-9: ITO/TPD(50 nm)/TST(30 nm)/TPBI(10 nm)/Alq(30 nm)/Mg:Ag(55 nm)/Ag(100 nm)

Example 45: tst-10: ITO/CuPc(10 nm)/TPD(50 nm)/TST(30 nm)/TPBI(40 nm)/Mg:Ag(55 nm)/Ag(100 nm)

Example 46: tst-11: ITO/CuPc(10 nm)/TPD(50 nm)/TST(30 nm)/TPBI(40 nm)/LiF (1 nm)/Ag(100 nm)

Example 47: psp-1: ITO/NPB(50 nm)/PSP(30 nm)/TPBI(40 nm)/Mg:Ag(55 nm)/Ag(100 nm)

Example 48: psp-2: ITO/NPB(50 nm)/PSP(30 nm)/TPBI(40 nm)/LiF(1 nm)/Al(100 nm)

Example 49: psp-3: ITO/CuPc(10 nm)/NPB(50 nm)/PSP(30 nm)//TPBI(40 nm)/Mg:Ag(55 nm)/Ag(100 nm)

Example 50: psp-4: ITO/CuPc(10 nm)/NPB(50 nm)/PSP(30 nm)/TPBI(40 nm)/ LiF(1 nm)/Al(100 nm)

Example 51: psp-5: ITO/TPD(50 nm)/PSP(30 nm)/TPBI(40 nm)/Mg:Ag(55 nm)/Ag(100 nm)

Example 52: psp-6: ITO/TPD(50 nm)/PSP(30 nm)/TPBI(40 nm)/LiF(1 nm)/Al(100 nm)

Example 53: psp-7: ITO/CuPc(10 nm)/TPD(50 nm)/PSP(30 nm)/TPBI(40 nm)/Mg:Ag(55 nm)/Ag(100 nm)

Example 54: psp-8: ITO/CuPc(10 nm)/TPD(50 nm)/PSP(30 nm)/TPBI(40 nm)/LiF(1 nm)/Al(100 nm)

Example 55: pst-1: ITO/NPB(50 nm)/PST(30 nm)/ TPBI(40 nm)/Mg:Ag(55 nm)/Ag(100 nm)

Example 56: pst-2: ITO/NPB(50 nm)/PST(30 nm)/TPBI(40 nm)/LiF(1 nm)/Al(100 nm)

Example 57: pst-3: ITO/CuPc(10 nm)/NPB(50 nm)/PST(30 nm)/TPBI(40 nm)/Mg:Ag(55 nm)/Ag(100 nm)

Example 58: pst-4: ITO/CuPc(10 nm)/NPB(50 nm)/PST(30 nm)/TPBI(40 nm)/LiF(I nm)/Al(100 nm)

Example 59: pst-5: ITO/m-MTDATA(10 nm)/NPB(50 nm)/PST(30 nm)/TPBI(40 nm)/Mg:Ag(55 nm)/Ag(100 nm)

Example 60: pst-6: ITO/m-MTDATA(10 nm)/NPB(50 nm)/PST(30 nm)/ TPBI(40 nm)/LiF(1 nm)/Al(100 nm)

Example 61: pst-7: ITO/TPD(50 nm)/PST(30 nm)/TPBI(40 nm)/Mg:Ag(55 nm)/Ag(100 nm)

Example 62: pst-8: ITO/TPD(50 nm)/PST(30 nm)/TPBI(40 nm)/LiF(1 nm)/Al(100 nm)

Example 63: pst-9: ITO/CuPc(10 nm)/TPD(50 nm)/PST(30 nm)/TPBI(40 nm)/Mg:Ag(55 nm)/Ag(100 nm)

Example 64: pst-10: ITO/CuPc(10 nm)/TPD(50 nm)/PST(30 nm)/TPBI(40 nm)/LiF(1 nm)/Al(100 nm)

TABLE 3
The properties of OLED devices using PT, PPT, PBT, TST,
PSP and PST as light emitting layer.

| | | | Maximum | | |
|---|---|---|---|---|---|
| | External | Maximum | current | CIE | |
| | quantum | Brightness | efficiency | coordinate | |
| | efficiency % | (cd/m²) | (cd/A) | (x, y) | |
| example | (V) | (V) | (V) | (8 V) | Color of light |
| Example 4 | 2.47(7.0) | 21801(18.5) | 4.03(7.0) | (0.15, 0.21) | blue |
| Example 5 | 2.60(6.5) | 24225(20.5) | 4.13(6.5) | (0.15, 0.19) | blue |
| Example 6 | 1.83(9.0) | 14593(19.5) | 3.11(9.0) | (0.16, 0.21) | blue |
| Example 7 | 2.35(6.0) | 23734(19.5) | 3.93(6.0) | (0.16, 0.22) | blue |
| Example 8 | 1.54(8.5) | 14460(18.5) | 3.01(8.5) | (0.17, 0.27) | blue |
| Example 9 | 1.64(10.0) | 14779(16.5) | 3.39(10.0) | (0.17, 0.29) | blue |
| Example 10 | 2.43(7.5) | 30148(19.0) | 4.98(7.5) | (0.17, 0.29) | blue |
| Example 11 | 2.13(6.5) | 18325(15.0) | 3.21(6.5) | (0.15, 0.20) | blue |
| Example 12 | 2.48(5.5) | 19498(18.0) | 3.66(5.5) | (0.15, 0.19) | blue |
| Example 13 | 3.79(8.50 | 29757(20.0) | 6.26(8.5) | (0.14, 0.20) | blue |
| Example 14 | 4.38(4.0) | 38751(19.5) | 6.33(4.0) | (0.15, 0.17) | blue |
| Example 15 | 3.49(7.5) | 27455(21.5) | 6.26(7.5) | (0.15, 0.22) | blue |
| Example 16 | 2.79(9.0) | 22359(18.5) | 3.89(9.0) | (0.14, 0.16) | blue |
| Example 17 | 3.89(8.5) | 64194(20.0) | 8.26(8.5) | (0.16, 0.27) | blue |
| Example 18 | 3.82(7.0) | 51833(17.5) | 7.31(7.0) | (0.15, 0.24) | blue |
| Example 19 | 4.59(3.5) | 57848(19.0) | 8.44(3.5) | (0.15, 0.24) | blue |
| Example 20 | 3.93(5.0) | 29301(20.0) | 7.31(5.0) | (0.16, 0.23) | blue |
| Example 21 | 4.57(4.0) | 39281(20.0) | 7.25(4.0) | (0.14, 0.19) | blue |
| Example 22 | 3.92(8.0) | 39966(15.5) | 6.42(7.5) | (0.15, 0.20) | blue |
| Example 23 | 4.25(5.0) | 29848(17.0) | 4.36(5.0) | (0.14, 0.11) | blue |
| Example 24 | 4.95(4.5) | 34002(21.5) | 4.80(4.5) | (0.14, 0.11) | blue |
| Example 25 | 4.08(7.0) | 32553(17.5) | 5.76(7.0) | (0.15, 0.17) | blue |
| Example 26 | 5.05(4.5) | 38549(16.5) | 6.32(4.5) | (0.15, 0.14) | blue |
| Example 27 | 3.05(9.0) | 25879(18.5) | 4.68(9.0) | (0.15, 0.18) | blue |
| Example 28 | 4.60(7.5) | 40979(18.5) | 6.19(8.0) | (0.15, 0.16) | blue |
| Example 29 | 5.23(7.0) | 41698(18.5) | 5.77(7.0) | (0.14, 0.12) | blue |
| Example 30 | 2.21(10.5) | 26171(17.5) | 3.34(11.0) | (0.15, 0.18) | blue |
| Example 31 | 2.78(6.5) | 25436(16.5) | 3.51(6.5) | (0.14, 0.14) | blue |
| Example 32 | 2.53(8.0) | 23862(18.0) | 3.73(8.0) | (0.15, 0.18) | blue |
| Example 33 | 2.62(5.5) | 27155(16.0) | 3.91(6.0) | (0.15, 0.18) | blue |
| Example 34 | 3.07(8.5) | 25191(17.0) | 4.28(8.5) | (0.14, 0.16) | blue |
| Example 35 | 3.28(7.5) | 25079(16.5) | 4.18(7.5) | (0.14, 0.15) | blue |
| Example 36 | 2.22(5.5) | 46486(17.0) | 6.37(5.5) | (0.20, 0.48) | blue green |
| Example 37 | 2.37(5.0) | 49664(20.5) | 7.34(5.0) | (0.24, 0.51) | blue green |
| Example 38 | 2.00(5.5) | 29190(19.0) | 5.70(5.5) | (0.20, 0.48) | blue green |
| Example 39 | 1.89(5.5) | 27117(20.5) | 5.12(5.5) | (0.19, 0.46) | blue green |
| Example 40 | 1.97(9.0) | 30843(21.0) | 6.51(7.5) | (0.25, 0.54) | blue green |
| Example 41 | 2.60(4.5) | 40405(20.0) | 8.45(4.5) | (0.24, 0.54) | blue green |
| Example 42 | 2.38(5.5) | 42865(18.0) | 6.54(5.5) | (0.19, 0.46) | blue |
| Example 43 | 2.93(5.0) | 45731(18.5) | 8.76(5.0) | (0.21, 0.50) | blue |
| Example 44 | 1.88(5.5) | 26472(19.5) | 5.05(5.5) | (0.19, 0.45) | blue |
| Example 45 | 1.73(7.5) | 27780(17.5) | 4.57(7.5) | (0.18, 0.45) | blue |
| Example 46 | 2.24(5.5) | 30139(16.0) | 6.21(5.5) | (0.19, 0.46) | blue |
| Example 47 | 1.60(7.0) | 42318(17.0) | 5.50(7.0) | (0.24, 0.61) | green |
| Example 48 | 1.79(6.0) | 48124(16.5) | 6.05(6.0) | (0.24, 0.60) | green |
| Example 49 | 1.63(7.5) | 44374(17.5) | 5.57(7.5) | (0.25, 0.60) | green |
| Example 50 | 1.72(5.5) | 42836(16.5) | 5.80(5.5) | (0.24, 0.60) | green |
| Example 51 | 1.41(9.0) | 39351(17.5) | 4.60(9.0) | (0.24, 0.58) | green |
| Example 52 | 1.50(8.0) | 41761(20.0) | 5.03(8.0) | (0.25, 0.59) | green |
| Example 53 | 1.97(8.0) | 44098(17.0) | 6.97(8.0) | (0.26, 0.61) | green |
| Example 54 | 2.29(6.0) | 46606(15.0) | 7.96(5.5) | (0.25, 0.61) | green |
| Example 55 | 1.76(7.0) | 54950(17.0) | 6.35(7.0) | (0.29, 0.60) | green |
| Example 56 | 2.13(5.0) | 68834(16.50 | 7.60(5.0) | (0.29, 06.0) | green |
| Example 57 | 2.14(7.5) | 61373(18.5) | 7.81(7.5) | (0.28, 0.61) | green |
| Example 58 | 2.36(5.0) | 70331(18.0) | 8.49(5.0) | (0.27, 0.61) | green |
| Example 59 | 2.91(10.0) | 65987(20.0) | 10.66(10.0) | (0.30, 0.61) | green |
| Example 60 | 3.10(8.0) | 72327(19.5) | 11.35(8.0) | (0.30, 0.61) | green |
| Example 61 | 1.81(7.5) | 52980(16.0) | 6.19(7.50 | (0.27, 0.59) | green |
| Example 62 | 2.20(4.5) | 60858(26.0) | 7.46(4.5) | (0.26, 0.59) | green |
| Example 63 | 1.94(7.0) | 49170(16.0) | 7.15(7.0) | (0.29, 0.61) | green |
| Example 64 | 2.38(5.0) | 45267(15.0) | 8.26(5.0) | (0.26, 0.60) | green |

The data in Table 3 showed that the organic luminescence device using the asymmetric compound of the present invention as the blue-light and green-light emitting layer showed good performance. After fabricating a device, PPT and PBT all exhibited excellent performance. Using PPT as an emitting layer, maximum brightness of the device was 64194 cd/m², external quantum efficiency was 4.59%, maximum current efficiency was 8.44 cd/A, and maximum power efficiency was 7.59 Im/W. Using PBT as an emitting layer, maximum brightness of the device was 41698 cd/m², external quantum efficiency over theoretical value was up to 5.23%, maximum current efficiency was 6.32cd/A, and maximum power efficiency was 4.89 Im/W. Because of the excellent blue-emitting materials of PPT and PBT, the PPT and PBT can be used in research related to white fluorescence. Using PST as light emitting layer, the glass transition temperature was 105° C. For the pst-6 in example 60, the device had a maximum brightness of 72327 cd/m², an external quantum efficiency of 3.10%, a maximum current efficiency of 11.35 cd/A, and a maximum power efficiency of 4.60 Im/W. The PST compound was also a good green-emitting material, which also can be used in research related to white fluorescence.

While the invention has been described by way of example and in terms of the preferred embodiments, it is to be understood that the invention is not limited to the disclosed embodiments. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A compound of formula □:
Ar¹—R¹—Ar² (I),
wherein Ar¹ and Ar² independently represent triphenylenyl or pyrenyl and R¹ represents a bond, aryl or heteroaryl.

2. The compound as claimed in claim 1, wherein aryl is selected from the group consisting of: phenyl, naphthyl, diphenyl, anthryl, pyrenyl, phenanthryl, fluorene, and other fused polycyclic phenyl.

3. The compound as claimed in claim 1, wherein heteroaryl is selected from the group consisting of: pyrane, pyrroline, furan, benzofuran, thiophene, benzothiophene, pyridine, quinoline, isoquinoline, pyrazine, pyrimidine, pyrazole, imidazole, indole, thiazole, isothiazole, oxazole, isoxazole, benzothiazole, benzoxazole, 1,2,4-triazole, 1,2,3-triazole, phenanthroline, and other heteroaryl.

4. The compound as claimed in claim 1, wherein Ar¹, Ar² and R¹ independently have one or more substituents selected from the group consisting of: hydrogen, halogen, aryl, halogen-substituted aryl, halogen-substituted aryl alkyl, haloalkyl-substituted aryl, haloalkyl-substituted aryl alkyl, aryl-substituted C1-C20 alkyl, electron donating group, electron withdrawing group, and heterocyclo-substituents.

5. The compound as claimed in claim 4, wherein the electron donating group comprises C1-C20 alkyl, C1-C20 cycloalkyl, C1-C20 alkoxy, C1-C20-substituted amino, or substituted aryl amino.

6. The compound as claimed in claim 4, wherein the electron withdrawing group comprises halogen, nitrous, nitro, carbonyl, cyano, or halogen-substituted C1-C20 alkyl.

7. The compound as claimed in claim 1, wherein R¹ is heteroaryl when Ar¹ and Ar² are the same.

8. The compound as claimed in claim 1, wherein:
(a) the compound is of formula (PT), when R¹ is a bond, 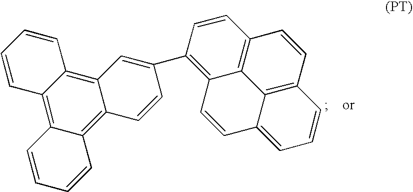
(b) the compound is of formula (PPT), when R¹ is phenyl, and Ar¹ is different from Ar², 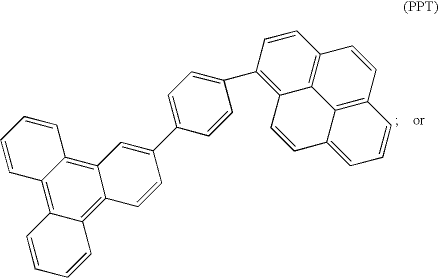
(c) the compound is of formula (PBT), when R¹ is biphenyl, and Ar¹ is different from Ar², 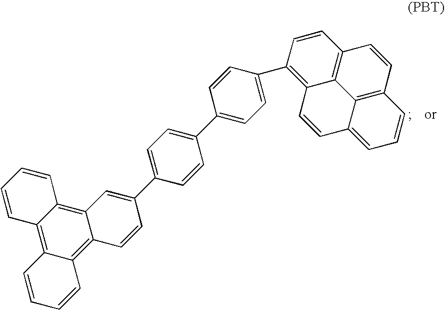
(d) the compound is of formula (TST), when R¹ is thiophene, and Ar¹ and Ar² are triphenylenyl, 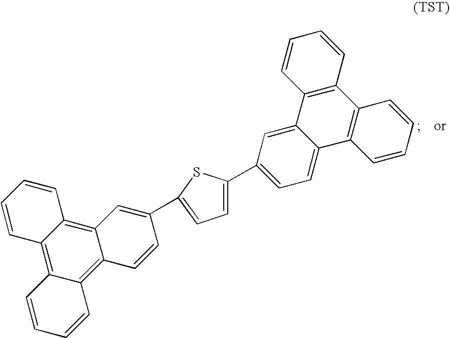
(e) the compound is of formula (PSP), when R¹ is thiophene, and Ar¹ and Ar² are pyrenyl, 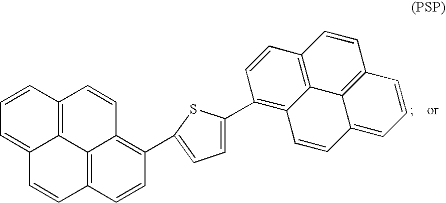
(f) the compound is of formula (PST), when R¹ is thiophene, and Ar¹ is different from Ar²,

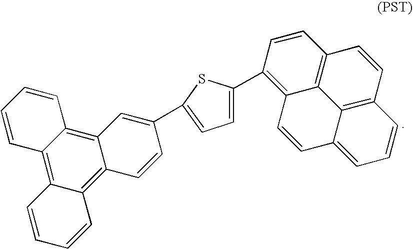

9. A process of preparing the compound of claim 1, comprising:
(a) reacting a compound of formula (□) with a compound of formula (□) to result in the compound of formula (□) when R¹ is a bond, 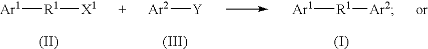
(b) reacting a compound of formula (□) with a compound of formula (□) to result in the compound of formula (□) when R¹ is aryl or heteroaryl and Ar¹ is different from Ar²;
(c) reacting a compound of formula (□) with a compound of formula (□) to result in the compound of formula (□) when R¹ is aryl or heteroaryl, and Ar¹ and Ar² are triphenylenyl, 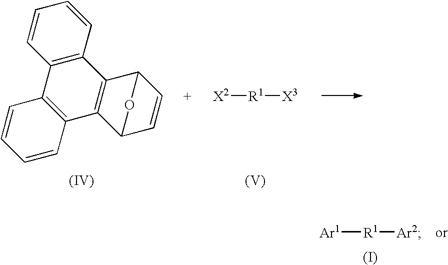
(d) reacting a compound of formula (□) with a compound of formula (□) to result in the compound of formula (□) when R¹ is aryl or heteroaryl, and Ar¹ and Ar² are pyrenyl, 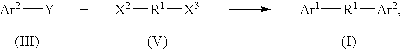
wherein X¹, X² and X³ are chlorine (Cl), bromine (Br) or iodine (I), and Y is boron hydroxide (B(OH)₂).

10. The process as claimed in claim 9, wherein the compound of formula (□) in step (b) is produced by reacting a compound of formula (□) with a compound of formula (□),

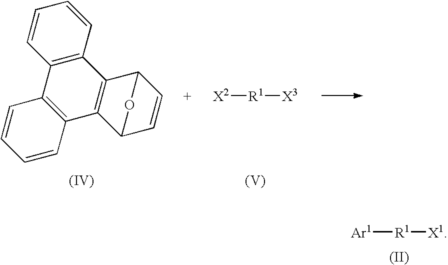

11. The process as claimed in claim 9, wherein the step (a), (b) and (d) are carried out by Suzuki coupling reaction.

12. The process as claimed in claim 9, wherein the step (c) is carried out by a coupling reaction.

13. The process as claimed in claim 10, wherein the reaction is carried out by a coupling reaction.

14. An organic electroluminescence device characterized by a light emitting layer comprising the compound of claim 1.

15. The device as claimed in claim 14, further comprising an anode, a hole transporting layer, an electron transporting layer, and a cathode.

16. The device as claimed in claim 15, further comprising a hole injection layer between the anode and the hole transporting layer.

17. The device as claimed in claim 15, the further comprising a hole blocking layer between the light emitting layer and the electron transporting layer.

18. The device as claimed in claim 14, wherein the device emits blue light, when R¹ is a bond or aryl.

19. The device as claimed in claim 14, wherein the device emits green light, when R¹ is heteroaryl, and Ar¹ and Ar² are not triphenylenyl at the same time.

20. The device as claimed in claim 14, wherein the device emits blue-green light, when R¹ is heteroaryl, Ar¹ and Ar² are triphenylenyl, and the hole transporting layer is N, N′-bis-phenyl-N, N′-bis-(1-naphthyl)-1,1′-biphenyl-4,4′-diamine (NPB).

21. The device as claimed in claim 14, wherein the device emits blue light, when R¹ is heteroaryl, Ar¹ and Ar² are triphenylenyl, and the hole transporting layer is N,N′-diphenyl-N, N′-bis-(3-methylphenyl)-1,1′biphenyl-4,4′-diamine (TPD).
